SEQUENCE LISTING (2 sequences)

SEQ ID NO: 1 - DNA, 20 bp, Synthetic construct
  source 1..20: mol_type = other DNA; note = Primer 27F
agagtttgatcmtggctcag

SEQ ID NO: 2 - DNA, 19 bp, Synthetic construct
  source 1..19: mol_type = other DNA; note = Primer 1492R
ggttaccttgttacgactt

(19) United States
(12) Patent Application Publication
(10) Pub. No.: US 2026/0242263 A1
(43) Pub. Date: Aug. 20, 2026

(54) BACILLUS SP. SDC-U1 STRAIN WITH QUORUM QUENCHING ACTIVITY, COMPOSITION FOR INHIBITING BIOFILM INCLUDING THE SAME AND SYSTEM FOR TREATING WATER USING THE SAME FOR MEMBRANE BIOREACTOR

(71) Applicants: Samsung Display Co., LTD., Yongin-si, Gyeonggi-do (KR); Foundation for Research and Business, Seoul National University of Science and Technology, Seoul (KR)

(72) Inventors: MYUNGHEE KIM, Asan-si, Chungcheongnam-do (KR); Hyun-Suk Oh, Seoul (KR); KEUMYONG KIM, Cheonan-si, Chungcheongnam-do (KR); BYUNGKOOK HWANG, Asan-si, Chungcheongnam-do (KR); Hyeok Kim, Seoul (KR); Abdolvahed Noori, Seoul (KR)

(21) Appl. No.: 19/663,713

(22) Filed: Apr. 30, 2026

Related U.S. Application Data
(62) Division of application No. 18/209,104, filed on Jun. 13, 2023.

(30) Foreign Application Priority Data
Jun. 13, 2022 (KR) 10-2022-0071579

(51) Int. Cl. C02F 3/34; A61K 35/742; C02F 3/12; C02F 101/34; C02F 101/38; C02F 103/34; C12N 1/205; C12N 11/10; C12R 1/07

(52) U.S. Cl. CPC C02F 3/342; A61K 35/742; C02F 3/1268; C12N 1/205; C12N 11/10; C02F 2101/34; C02F 2101/38; C02F 2103/34; C02F 2303/20; C12R 2001/07

(57) ABSTRACT

Bacillus sp. SDC-U1 strain deposited to Korean Collection for Type Cultures with Accession No. KCTC 14857BP has quorum quenching activity.

This application claims priority to Korean Patent Application No. 10-2022-0071579, filed on Jun. 13, 2022, and all the benefits accruing therefrom under 35 U.S.C. § 119, the content of which in its entirety is herein incorporated by reference.

### SEQUENCE LISTING

The Instant Application contains a Sequence Listing which has been submitted electronically in XML format and is hereby incorporated by reference in its entirety. Said XML copy, created on Aug. 26, 2025, is named “21C1410US” and is 3,365 bytes in size. The Sequence Listing does not go beyond the disclosure in the application as filed.

### BACKGROUND

### 1. Field

Embodiments relate to a strain with quorum quenching activity. More particularly, embodiments relate to a strain with quorum quenching activity, a composition including the strain for inhibiting a biofilm, a system for treating water using the composition for a membrane bioreactor.

### 2. Description of the Related Art

A membrane bioreactor (MBR) maybe used for a water treatment technology that filters wastewater, which includes sewage water, through a membrane to remove floating matters, microorganisms or the like after the waster mater is biologically decomposed.

### SUMMARY

In a membrane bioreactor (MBR), a biofilm may be formed on a surface of a membrane thereof by microorganisms existing in wastewater. As a result, water permeability, cleaning period or the like of the membrane may be reduced, and performance of the bioreactor may be deteriorated. In order to inhibit quorum sensing mechanism that forms the biofilm, microorganisms that may enzymatically degrade acyl-homoserine lactone (AHL) functioning as signaling molecules are being researched.

Embodiments provide a strain having with quorum quenching activity.

Embodiments provide a composition for inhibiting a biofilm, which includes the strain.

Embodiments provide a water-treating system using the strain for a membrane bioreactor.

According to an embodiment, Bacillus sp. SDC-U1 strain having quorum quenching activity and deposited to Korean Collection for Type Cultures with Accession No. KCTC 14857BP is provided.

In an embodiment, the Bacillus sp. SDC-U1 strain produces acyl-homoserine lactone (AHL)-degrading enzymes.

In an embodiment, the Bacillus sp. SDC-U1 strain inhibits a biofilm formed by a strain using AHL as a signaling molecule.

According to an embodiment, a composition for inhibiting a biofilm formation includes Bacillus sp. SDC-U1 strain having quorum quenching activity and being deposited to Korean Collection for Type Cultures with Accession No. KCTC 14857BP or a cultured product thereof.

In an embodiment, the cultured product includes at least one selected from cultures, debris and fraction of the Bacillus sp. SDC-U1 strain.

In an embodiment, the composition further includes a carrier.

According to an embodiment, a water treatment system includes a reactor into which wastewater flows, a membrane including at least a portion dipped in the wastewater in the reactor, and a microorganism-fixing medium disposed in the reactor, where the microorganism-fixing medium fixes Bacillus sp. SDC-U1 strain having quorum quenching activity and deposited to Korean Collection for Type Cultures with Accession No. KCTC 14857BP or a cultured product thereof.

In an embodiment, the wastewater includes tetramethylammonium hydroxide (TMAH).

In an embodiment, activated sludge including microorganisms is provided in the reactor.

In an embodiment, the wastewater after treated in the reactor is discharged from the reactor through the membrane.

In an embodiment, the microorganism-fixing medium includes a fixing bead.

In an embodiment, the microorganism-fixing medium includes an alginate bead.

According to embodiments, a biofilm formation may be inhibited by a strain having quorum quenching activity.

In such embodiments, the strain may not be inactivated in a condition including TMAH. Thus, increase of transmembrane pressure due to a biofilm formation in a system for treating wastewater including a hardly degradable substances may be delayed. Thus, performance of treating wastewater may be improved, and a membrane-cleaning period may be increased.

### BRIEF DESCRIPTION OF THE DRAWINGS

The above and other features of one or more embodiments of the invention will be more clearly understood from the following detailed description taken in conjunction with the accompanying drawings.

FIG. 1 is a phylogenetic tree of Bacillus sp. SDC-U1 strain.

FIG. 2 is a transmission electron microscope (TEM) image showing Bacillus sp. SDC-U1 strain.

FIG. 3 schematically shows a water-treatment system according to an embodiment.

FIG. 4 is a graph showing the QQ rate coefficients of Bacillus sp. SDC-U1 strain to AHL.

FIG. 5 is a graph showing optical densities (OD₆₀₀) of the Bacillus sp. SDC-U1 strain solution cultured in the anaerobic chamber.

FIG. 6 is a graph showing concentrations of C8-HSL in the Bacillus sp. SDC-U1 strain solution cultured in the anaerobic chamber.

FIG. 7 is a graph showing concentrations of C8-HSL in the Bacillus sp. SDC-U1 strain solution and in the SDC-D14 strain solution, which were cultured under tetramethylammonium hydroxide (TMAH)-containing conditions.

FIG. 8 is a graph showing amounts and reduction of the biofilms formed from the cultured solution of PAO1 and the co-cultured solution of PAO1/SDC-U1.

FIG. 9 is a graph showing amounts and reduction of the biofilms formed from the cultured solution of the activated sludge (A.S.) and the co-cultured solution of A.S./SDC-U1.

FIG. 10 is a graph showing the transmembrane pressure (TMP) of the membrane bioreactor that was operated with the fixing beads (Alg bead) combined with SDC-U1 strain.

### DETAILED DESCRIPTION

The invention now will be described more fully hereinafter with reference to the accompanying drawings, in which various embodiments are shown. This invention may, however, be embodied in many different forms, and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, “a”, “an,” “the,” and “at least one” do not denote a limitation of quantity, and are intended to include both the singular and plural, unless the context clearly indicates otherwise. For example, “an element” has the same meaning as “at least one element,” unless the context clearly indicates otherwise. “At least one” is not to be construed as limiting “a” or “an.” “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Hereinafter, “biofilm” may mean aggregation of microorganisms formed in a polymeric matrix secreted by the microorganisms thereby forming a film shape on a solid surface.

An embodiment provides Bacillus sp. SDC-U1 strain that has quorum quenching activity and has been deposited with Accession No. KCTC 14857BP to Korean Collection for Type Cultures on Jan. 20, 2022.

Activated sludge of a facility (Samsung Display) for treating wastewater from processes for manufacturing a display device was enrichment-cultured through a medium including an acyl-homoserine lactone (AHL) mixture including N-hexanoyl-homoserin lactone (C6-HSL), N-3-oxo-hexanoyl-HSL (3-oxo-C6-HSL), N-dodecanoyl HSL (C12-HSL) and N-3-oxo-dodecanoyl HSL (3-oxo-C12-HSL) as the sole carbon source. The SDC-U1 strain having superior activity was selected from pure isolates obtained from the enrichment cultures.

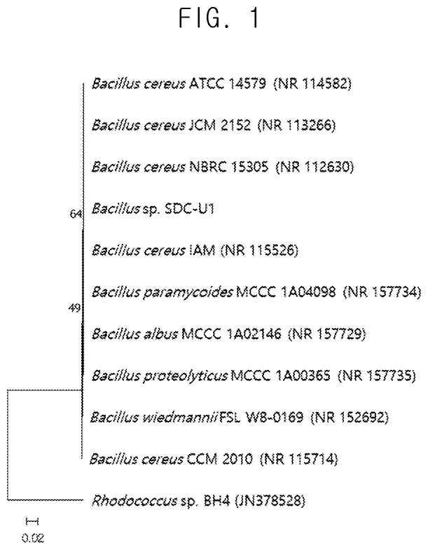

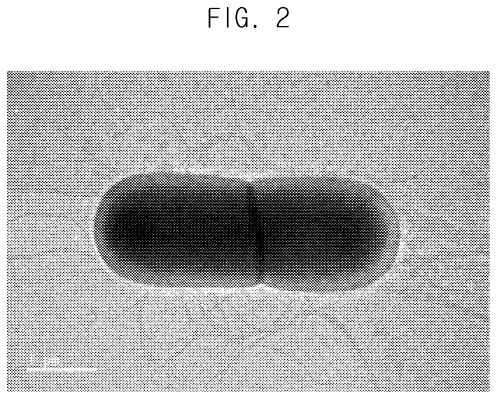

FIG. 1 is a phylogenetic tree of Bacillus sp. SDC-U1 strain. FIG. 2 is a transmission electron microscope (TEM) image showing Bacillus sp. SDC-U1 strain.

The SDC-U1 strain were sequenced through a polymerase chain reaction (PCR) amplification using a universal primer set of 27F (5′-AGA GTT TGA TCM TGG CTC AG-3′n (SEQ ID NO:1)) and 1492R (5′-GGT TAC CTT GTT ACG ACT T-3′ (SEQ ID NO: 2)) for bacterial identification. Referring to FIG. 1, the 16S rDNA analysis revealed that the SDC-U1 strain belonged to Bacillus sp. and had high similarity with Bacillus cereus, Bacillus paramycoides, Bacillus albus, Bacillus proteolyticus or the like (more than 99.0%).

Bacillus sp. SDC-U1 strain was observed using a transmission electron microscope to obtain morphological characteristics. Referring to FIG. 2, it is shown that Bacillus sp. SDC-U1 strain has a bar shape like known strains belonging to Bacillus sp. and a cell shape with about 1.2 μm of a width and about 3 μm of a length.

In an embodiment, Bacillus sp. SDC-U1 strain may be obtained from activated sludge of a facility for treating industrial wastewater including tetramethylammonium hydroxide (TMAH).

Bacillus sp. SDC-U1 strain may produce AHL-degrading enzymes to have quorum quenching activity. Thus, a biofilm formation, e.g., a biofilm formed by strains using AHL as signaling molecules, may be inhibited or controlled by Bacillus sp. SDC-U1 strain.

For example, acyl-homoserin lactones that may be degraded by Bacillus sp. SDC-U1 strain may include C6-HSL, N-octanoyl-HSL (C8-HSL), N-decanoyl-HSL (C10-HSL), C12-HSL, 3-oxo-C6-HSL, 3-oxo-C8-HSL, 3-oxo-C10-HSL, 3-oxo-C12-HSL or the like.

For example, the strains using AHL as signaling molecules may include C. violaceum, Y. enterocolitica, P. aeruginosa or the like.

According to an embodiment, a biofilm formation may be inhibited by Bacillus sp. SDC-U1 strain in processes for treating wastewater. In an embodiment, for example, the processes for treating wastewater may be performed in a membrane bioreactor, and the wastewater or activated sludge in the membrane bioreactor may include the strains using AHL as signaling molecules. In such an embodiment, Bacillus sp. SDC-U1 strain or a composition thereof is provided to the strains using AHL as signaling molecules, such that a biofilm formation may be inhibited.

For example, a microorganism-fixing medium for fixing Bacillus sp. SDC-U1 strain may be provided in the reactor to treat the strains using AHL as signaling molecules with Bacillus sp. SDC-U1 strain or a composition thereof, Bacillus sp. SDC-U1 strain may be fixed at a membrane, and wastewater or activated sludge may be inoculated with Bacillus sp. SDC-U1 strain.

An embodiment provides a composition for inhibiting a biofilm formation. The composition includes Bacillus sp. SDC-U1 strain or cultured products thereof. The cultured products may include at least one selected from cultures, debris and fraction of Bacillus sp. SDC-U1 strain.

The composition for inhibiting a biofilm formation may include a carrier to carry microorganisms. In an embodiment, for example, the carrier may have a particle shape. In an embodiment, for example, the carrier may include an organic material such as a polymer, an inorganic material such as silica, metals or the like, or an organic-inorganic composite.

In an embodiment, for example, Bacillus sp. SDC-U1 strain may be used for treating industrial wastewater including TMAH. However, embodiments are not limited thereto. In an embodiment, for example, Bacillus sp. SDC-U1 strain may be used to treat industrial wastewater including a material that may be hardly degradable. In an embodiment, Bacillus sp. SDC-U1 strain may be used to treat various wastewaters such as sewage water as well as industrial wastewater.

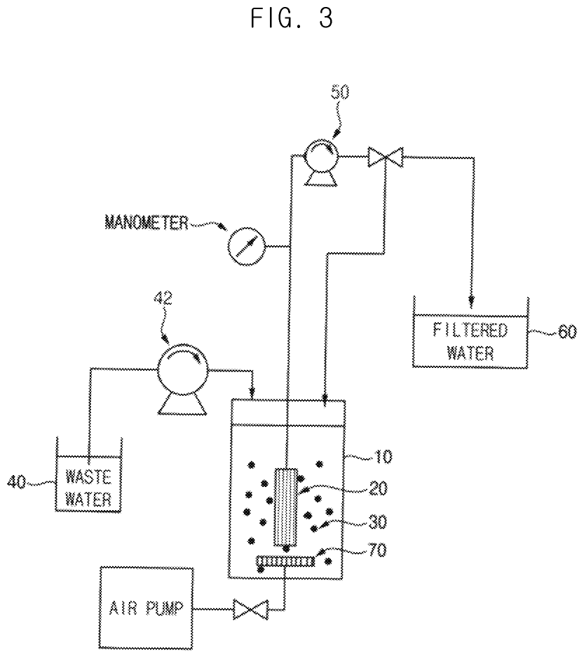

FIG. 3 schematically shows a water-treatment system according to an embodiment.

Referring to FIG. 3, an embodiment of the water-treatment system includes a reactor 10, a membrane 20 disposed in the reactor 10 and a microorganism-fixing medium 30.

Wastewater is provided in the reactor 10. In an embodiment, the wastewater provided in the reactor 10 may be industrial wastewater including TMAH.

The reactor 10 may be connected to a waste-water storage part 40 that stores wastewater. In an embodiment, for example, the wastewater may be provided in the reactor 10 by an inflow pump 42 or the like. Microorganisms exist in the reactor 10. The microorganisms may include bacteria, fungus, algae or the like. In an embodiment, for example, the microorganisms may be provided by activated sludge in the reactor 10.

As the wastewater is biologically degraded in the reactor 10, contaminants in the wastewater may be degraded. The treated (degraded) wastewater may be separated from floating matters, microorganisms or the like through the membrane 20. In an embodiment, for example, at least a portion of the membrane 20 may be dipped in the wastewater. The membrane 20 may be connected to a suction pump 50. The wastewater may be discharged from the reactor 10 through the membrane 20. The filtered water having passed through the membrane 20 may move to a treated-water storage part 60 or the like. The filtered water may partially move back to the reactor 10.

In an embodiment, an aeration apparatus 70 may be disposed in the reactor 10. The aeration apparatus 70 may provide the wastewater with air bubbles. The air bubbles may accelerate biological degradation of the wastewater.

In an embodiment, the membrane 20 may be a hollow fiber membrane. In an embodiment, for example, the hollow fiber membrane may include polyvinylidene fluoride (PVDF), polytetrafluoroethylene (PTFE), polyacrylonitrile (PAN), polyamide (PA), polyimide (PI), polysulfone (PS), polyethersulfone (PES), polyetherimide (PEI), polyethylene (PE), polyetheretherketone (PEEK), sulfonated polyetheretherketone (SPEEK), polybenzimidazole (PBI), poly(vinyl alcohol (PVA), polyvinylchloride (PVC), poly(trimethylsilyl propyne) (PTMSP), poly(trimethylgermylpropyne) (PTMGP), polymethylpentene (PMP), polydimethylsiloxane (PDMS), polycarbonate (PC), poly(ethylene oxide) (PEO), polyphenylene oxide (PPO), chitosan, poly(acrylic acid) (PAA), poly(sodium styrenesulfonate) (PSS), poly(vinyl sulfate) (PVS), polypropylene (PP), polypyrrole (PPy), polyphosphazene (PPz), polyurethane (PU), cellulose acetate, nitrocellulose, cellulose esters, ethyl cellulose or the like. However, embodiments are not limited thereto. In an alternative embodiment, for example, the membrane 20 may include inorganic composite including ceramic or the like.

The microorganism-fixing medium 30 may be disposed in the reactor 10. The microorganism-fixing medium 30 fixes microorganisms having quorum quenching activity. In an embodiment, the microorganism-fixing medium 30 may include a fixing bead.

In an embodiment, the microorganisms having quorum quenching activity include Bacillus sp. SDC-U1 strain that has been deposited with Accession No. KCTC 14857BP.

In an embodiment, the fixing bead may include an alginate bead. The alginate bead may have flexibility and a porous structure.

In an embodiment, for example, after an alginate solution and microorganism or cultured products thereof are mixed, the mixture may be dropped in a solution including metal ions to obtain a bead having a spherical shape.

In an embodiment, for example, the alginate solution includes alginate. In an embodiment, the alginate may include sodium alginate, potassium alginate, ammonium alginate, polyethyleneglycol-alginate or the like, for example.

The alginate solution may further include a polymer such as polyvinyl alcohol (PVA) or the like.

The metal ion solution may include metal ions with 2 valances. In an embodiment, for example, the metal ion solution may be obtained by dissolution of CaCl₂), MgCl₂, SrCl₂, SnCl₂, FeCl₂, CuCl₂, BaCl₂, CoCl₂, NiCl₂ or the like.

Embodiments are not limited to the microorganism-fixing medium using the fixing bead. In an alternative embodiment, for example, a microorganism-fixing medium may be coupled to the membrane 20, or the microorganisms having quorum quenching activity may be directly fixed at the membrane 20.

Hereinafter, embodiments of the invention will be described with reference to specific examples and experiments. However, the examples and experiments are described to explain effects of embodiments of the invention, and embodiments are not limited thereto.

### Isolation of Strains

A pellet, which had been obtained from an activated sludge of a facility of Samsung Display for treating wastewater from processes for manufacturing a display device, was re-suspended in a saline solution. The concentration of TMAH in the wastewater was 50-200 mg/L. 200 μl of the re-suspended solution was mixed with 160 μl of a minimum medium and 40 μl of AHL mixture (C6-HSL 1.5 mM, 3-oxo-C6-HSL 1.5 mM, C12-HSL 0.75 mM and 3-oxo-C12-HSL 0.75 mM) as a carbon source and HCl was added thereto such that the pH was 5.5, thereby preparing a medium.

After the medium was incubated, strains that formed a single colony having a distinct morphology were isolated.

### Identification of Strains

The isolated strains were sequenced through a PCR amplification using a universal primer set of 27F (5′-AGA GTT TGA TCM TGG CTC AG-3′ (SEQ ID NO:1)) and 1492R (5′-GGT TAC CTT GTT ACG ACT T-3′ (SEQ IS NO: 2)) for bacterial identification. Referring back to FIG. 1, the 16S rDNA analysis revealed that the SDC-U1 strain (NCBI Accession No. OM327595) belonged to Bacillus sp. and had high similarity with Bacillus cereus, Bacillus paramycoides, Bacillus albus, Bacillus proteolyticus or the like (more than 99.0%).

### Experiment: Degradation of AHL

The overnight cultures of the isolated strains were fractionated to obtain whole cells. The solid cell pellets were washed twice and re-suspended in Tris-HCl buffer (50 mM, pH 7.0) to obtain an OD₆₀₀ of 0.5. The cell resuspension in Tris-HCl buffer (to test whole-cell quorum quenching activity) was mixed with AHL (C6-HSL, C8-HSL, C10-HSL, C12-HSL, 3-oxo-C6-HSL, 3-oxo-C8-HSL, 3-oxo-C10-HSL and 3-oxo-C12-HSL) to a final concentration of 200 nM and incubated in a shaker at 180 rpm and 30° C. Thereafter, concentrations of AHL over time were measured using Agrobacterium tumefaciens A136 as a reporter strain via a luminescence method with a microplate reader (Synergy HTX, Biotek®), and the quorum quenching (QQ) rate coefficient was obtained therefrom. The QQ rate coefficient (k) was expressed as a pseudo-first-order function according to the degradation rate of the AHL signal molecule at time t, and calculated by the following.

$$\frac{d [ AHL ]}{dt} = k [ AHL ]$$

$$ln \frac{{[ AHL ]}_{t}}{{[ AHL ]}_{0}} = - kt$$

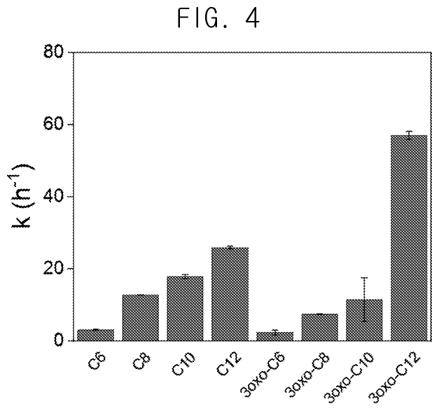

FIG. 4 is a graph showing the QQ rate coefficients of Bacillus sp. SDC-U1 strain to AHL. Referring to FIG. 4, it is shown that Bacillus sp. SDC-U1 strain degraded various AHLs well and that the QQ rate coefficients (k) of Bacillus sp. SDC-U1 strain to AHL was increased as a length of an acyl group in the AHLs was increased.

### Experiment: Growth Under Anaerobic Conditions

QQ activity under anaerobic conditions was tested to determine whether the isolated strains were facultative and could degrade the AHL signal molecule in the absence of dissolved oxygen (DO). Bacillus sp. SDC-U1 strain solution (OD₆₀₀ 1.0) and Tris-HCl buffer solution (50 mM, pH 7.0) containing the signal molecule C8-HSL (400 nM) were prepared separately in serum bottles and sealed with rubber stoppers and aluminum crimp caps. The headspace of the solutions was purged with nitrogen for 30-40 minutes until the DO levels in the solution reached below 0.05 mg/L inside an anaerobic chamber (Coy Lab Product, Inc., USA). Thereafter, 25 mL of the bacterial and C8-HSL solutions were mixed in a new serum bottle and placed inside the anaerobic chamber at 30° C. while being agitated with a magnetic bar (70 rpm) and purged continuously with nitrogen gas. The DO levels were measured using a digital DO meter (HI-2040 edge Hybrid Multiparameter DO Meter, Hanna, USA), and concentrations of C8-HSL over time were measured using Agrobacterium tumefaciens A136 as a reporter strain via a luminescence method with a microplate reader (Synergy HTX, Biotek®).

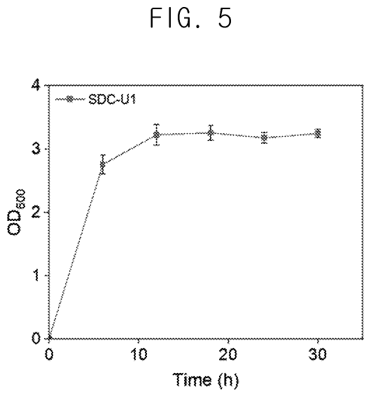

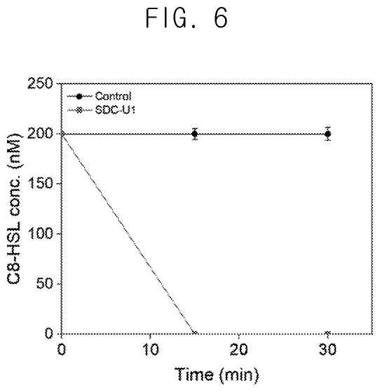

FIG. 5 is a graph showing optical densities (OD₆₀₀) of the Bacillus sp. SDC-U1 strain solution cultured in the anaerobic chamber. FIG. 6 is a graph showing concentrations of C8-HSL in the Bacillus sp. SDC-U1 strain solution cultured in the anaerobic chamber.

Referring to FIG. 5, Bacillus sp. SDC-U1 strain grew well under anaerobic conditions. Thus, it is shown that Bacillus sp. SDC-U1 strain is a facultative anaerobe.

Referring to FIG. 6, Bacillus sp. SDC-U1 strain degraded AHL well under anaerobic conditions.

### Experiment: Degradation of AHL Under TMAH-Containing Conditions

The overnight cultures of the isolated strains were fractionated into the supernatant of the cultured broth and whole cells. The solid cell pellets were washed twice and re-suspended in Tris-HCl buffer (50 mM, pH 7.0) to obtain an OD₆₀₀ of 0.5. The cell resuspension in Tris-HCl buffer (to test whole-cell QQ activity) was mixed with C8-HSL to a final concentration of 200 nM and incubated in a shaker at 180 rpm and 30° C. To test affection of toxic chemicals in industrial wastewater, TMAH (20 mg/L, 100 mg/L and 500 mg/L) was added to the incubated solution. Thereafter, concentrations of C8-HSL over time were measured using Agrobacterium tumefaciens A136 as a reporter strain via a luminescence method with a microplate reader (Synergy HTX, Biotek®).

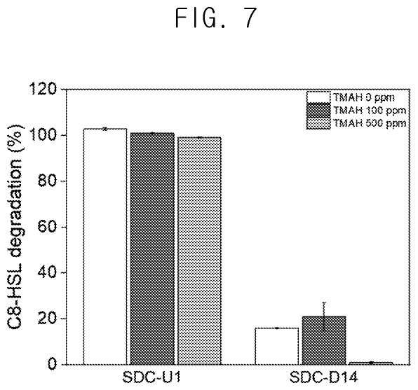

FIG. 7 is a graph showing concentrations of C8-HSL in the Bacillus sp. SDC-U1 strain solution and in the SDC-D14 strain solution, which were cultured under TMAH-containing conditions.

Referring to FIG. 7, the QQ activity of Bacillus sp. SDC-U1 strain was not reduced by TMAH. However, the QQ activity of SDC-D14 strain was substantially reduced when the concentration of TMAH was 500 mg/L.

SDC-14 was cultured and isolated from the same activated sludge as Bacillus sp. SDC-U1 strain. As a result of a sequence analysis through a same method as Bacillus sp. SDC-U1 strain, it was revealed that SDC-D14 (NCBI Accession No. OM327600) belonged to Stenotrophomonas sp. and had high similarity with Stenotrophomonas pavanii strain LMG 25,348 (99.72%).

### Experiment: Inhibition of Biofilm Formation

The impact of the isolated strains on biofilm formation of PAO1 or activated sludge was assessed through 24-well microtiter plate assays. The isolated strain (Bacillus sp. SDC-U1) and the biofilm-forming quorum sensing (QS) bacterium P. aeruginosa PAO1 (ATCC 15692) were grown in Luria-Bertani (LB) broth overnight and centrifuged at 4,500 rpm for 10 minutes. Then, the concentration of PAO1 was adjusted to OD₆₀₀ of 0.03, and different concentrations of Bacillus sp. SDC-U1 at an OD₆₀₀ of 0.01, 0.03, and 0.09 were co-cultured at a fixed volume, leading to cell ratios between PAO1 and Bacillus sp. SDC-U1 of 1:0.33, 1:1, and 1:3, respectively. Additionally, the activated sludge collected from the membrane bioreactor (Samsung Display) treating wastewater from process for manufacturing a display device was co-cultured with Bacillus sp. SDC-U1 at the same cell ratio as above.

The LB broth was replaced with real wastewater containing TMAH to simulate a more realistic situation.

The co-cultured solutions (1 mL) were placed in the wells of a 24-well microtiter plate and incubated for 6 hours at 30° C. with shaking (180 rpm). The biofilms that formed on the wells were quantified using crystal violet (CV) assays. The biofilm remaining in the wells was stained using 0.1% (0.1% w/v) CV for 20 minutes and washed two times with PBS buffer. Thereafter, the wells were destained with 99.9% ethanol and the quantity of CV in the solution was determined at OD₅₅₀ using a microplate reader. A sample with only P. aeruginosa PAO1 or activated sludge was used as a control.

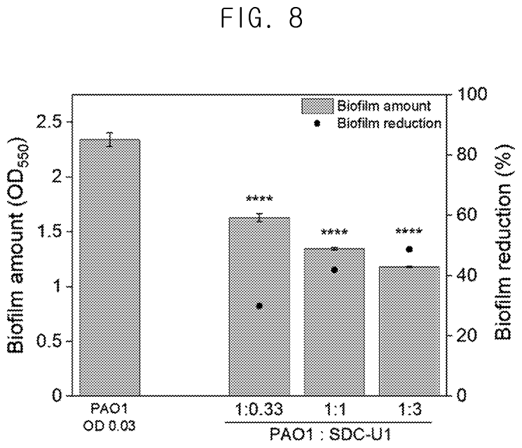

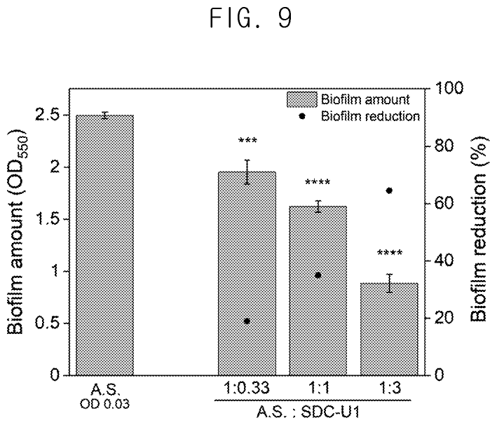

FIG. 8 is a graph showing amounts and reduction of the biofilms formed from the cultured solution of PAO1 and the co-cultured solution of PAO1/SDC-U1. FIG. 9 is a graph showing amounts and reduction of the biofilms formed from the cultured solution of the activated sludge (A.S.) and the co-cultured solution of A.S./SDC-U1.

Referring to FIGS. 8 and 9, as a ratio of SDC-U1 strain was increased, effects of inhibiting biofilm formations was increased as well.

### Experiment: Measurement of Transmembrane Pressure (TMP)

Concentrate of the cultured solution of SDC-U1 strain was mixed with Na-alginate (2%) and then dropped in CaCl₂) solution to obtain fixing beads combined with SDC-U1 strain. The fixing beads were provided in a laboratory-level membrane bioreactor, which was operated according to the following Table 1, and a transmembrane pressure of the membrane bioreactor over time was measured.

TABLE 1
Operation conditions

| | |
|---|---|
| Inoculation sludge | Activated sludge obtained from the |
| | membrane bioreactor of Samsung Display |
| Inflow wastewater | Wastewater flowing into the membrane |
| | bioreactor of Samsung Display |
| Volume of reactor | 1.9 L |
| HRT (Hydraulic | 32 h |
| Retention Time) | |
| SRT (Solids Retention Time) | 95 d |
| Flux | 16 LMG |
| MLSS | ~5,000 mg/L |
| Membrane | Material: PVDF, Nominal |
| | pore size: 0.03 μm |
| Amount of beads | 1.5% (v/v) |
| Concentration of TMAH in | 120-130 mg/L |
| Inflow wastewater | |
| Concentration of TMAH in | 7 mg/L |
| reactor | |

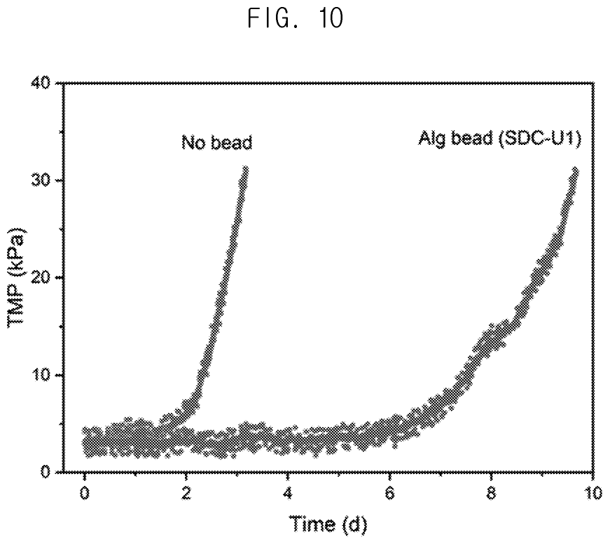

FIG. 10 is a graph showing the transmembrane pressure (TMP) of the membrane bioreactor that was operated with the fixing beads (Alg bead) combined with SDC-U1 strain.

Referring to FIG. 10, it is shown that the membrane-cleaning period (time lapse until TMP became 30 kPa) of the membrane bioreactor that was operated with the Alg bead was three times longer than that of comparative example (No bead).

Embodiments may be used for inhibiting a biofilm formation or a biofilm formed by quorum sensing. Embodiments may be used for treating wastewater, sewage water or the like, for example.

The invention should not be construed as being limited to the embodiments set forth herein. Rather, these embodiments are provided so that this disclosure will be thorough and complete and will fully convey the concept of the invention to those skilled in the art.

While the invention has been particularly shown and described with reference to embodiments thereof, it will be understood by those of ordinary skill in the art that various changes in form and details may be made therein without departing from the spirit or scope of the invention as defined by the following claims.

1-6. (canceled)

7. A system for treating water, comprising
a reactor into which wastewater flows;
a membrane including at least a portion dipped in the wastewater in the reactor; and
a microorganism-fixing medium disposed in the reactor, wherein microorganism-fixing medium fixes Bacillus sp. SDC-U1 strain having quorum quenching activity and deposited to Korean Collection for Type Cultures with Accession No. KCTC 14857BP or a cultured product thereof.

8. The system of claim 7, wherein the wastewater includes tetramethylammonium hydroxide (TMAH).

9. The system of claim 7, wherein activated sludge including microorganisms is provided in the reactor.

10. The system of claim 7, wherein the wastewater after treated in the reactor is discharged from the reactor through the membrane.

11. The system of claim 7, wherein the Bacillus sp. SDC-U1 strain produces acyl-homoserine lactone (AHL)-degrading enzymes.

12. The system of claim 7, wherein the Bacillus sp. SDC-U1 strain inhibits a formation of a biofilm, which is formed by a strain using AHL as a signaling molecule.

13. The system of claim 7, wherein the microorganism-fixing medium includes a fixing bead.

14. The system of claim 7, wherein the microorganism-fixing medium includes an alginate bead.
